(19) United States
(12) Patent Application Publication
(10) Pub. No.: US 2021/0272659 A1
(43) Pub. Date: Sep. 2, 2021

(54) SYSTEM TO PROVIDE SHARED DECISION MAKING FOR PATIENT TREATMENT OPTIONS

(71) Applicant: Minas Chrysopoulo, San Antonio, TX (US)

(72) Inventor: Minas Chrysopoulo, San Antonio, TX (US)

(21) Appl. No.: 17/274,052

(22) PCT Filed: Sep. 5, 2019
(86) PCT No.: PCT/US2019/049647

§ 371 Date: Mar. 5, 2021

Related U.S. Application Data
(60) Provisional application No. 62/728,231, filed on Sep. 7, 2018.

(51) Int. Cl. G16H 10/20; G16H 50/20; G16H 50/30; G16H 10/60; G16H 80/00

(52) U.S. Cl. CPC G16H 10/20; G16H 50/20; G16H 80/00; G16H 10/60; G16H 50/30

(57) ABSTRACT

Systems, apparatus, and methods for providing treatment recommendations for a disease state to a user interface are described. An initial response data regarding the disease state is received from the user interface. The initial response data is processed through an interface model to determine a series of next questions. The series of next questions is provided to the user interface. Subsequent response data for the series of next questions is received from the user interface. The initial response data and the subsequent response data are processed through a shared decision making engine to determine the treatment recommendations for the disease state from a plurality of treatment options for the disease state based on a weighted matrix, the weighted matrix including combinations of answers weighted according to relevance factors for treatment options for the disease state. The treatment recommendations for the disease state is provided to the user interface.

### CROSS-REFERENCE TO RELATED APPLICATION

This application claims the benefit of priority to U.S. Provisional Application No. 62/728,231 filed Sep. 7, 2018, the disclosure of which is incorporated herein by reference in its entirety.

### BACKGROUND

Shared decision making (SDM), which can be applied to any disease state, is the process by which a user's clinical situation, personal preferences and values, and evidence-based medicine are considered and weighted according to a trained decision making model to provide a recommendation that is best for the patient. Unfortunately, in the clinical setting, patients are often excluded from important discussions concerning their treatment and frequently feel like they are being left “in the dark.” In addition, healthcare cost constraints increasingly limit face-to-face time between patients and their physicians. As a result, many patients are overwhelmed attempting to navigate unfamiliar medical information on their own.

One important aspect of patient-centered care is the active engagement of patients. As such, SDM is a key component of patient-centered health care. The use of patient decision aids has been shown to not only aid in SDM but also improve patient education, improve patient perception of associated risks of therapy, increase the number of decisions that are consistent with patients' values, reduce the level of decisional conflict for patients, and decrease the number of patients who remain undecided.

### SUMMARY

Implementations of the present disclosure are generally directed to a shared decision making system employed to provide treatment options for patients. The described system employs both machine learning and artificial intelligence in conjunction with a shared decision making engine to provide users (e.g., patients) a customized treatment recommendation for a specific clinical problem or disease state, such as surgical treatment options for breast cancer, that can be broken down into its component subtopics.

In a general implementation, systems, apparatus, and methods for providing treatment recommendations for a disease state to a user interface. An initial response data regarding the disease state is received from the user interface. The initial response data is processed through an interface model to determine a series of next questions. The series of next questions is provided to the user interface. Subsequent response data for the series of next questions is received from the user interface. The initial response data and the subsequent response data are processed through a shared decision making engine to determine the treatment recommendations for the disease state from a plurality of treatment options for the disease state based on a weighted matrix, the weighted matrix including combinations of answers weighted according to relevance factors for treatment options for the disease state. The treatment recommendations for the disease state is provided to the user interface.

In another general implementation, one or more non-transitory computer-readable storage media coupled to one or more processors and having instructions stored thereon which, when executed by the one or more processors, cause the one or more processors to perform operations that include receiving, from a user interface, an initial response data regarding a disease state. The initial response data is processed through an interface model to determine a series of next questions. The series of next questions is provided to the user interface. Subsequent response data for the series of next questions is received from the user interface. The initial response data and the subsequent response data are processed through a shared decision making engine to determine the treatment recommendations for the disease state from a plurality of treatment options for the disease state based on a weighted matrix, the weighted matrix including combinations of answers weighted according to relevance factors for treatment options for the disease state. The treatment recommendations for the disease state is provided to the user interface.

In yet another general implementation, a display device; a system includes one or more processors; and a computer-readable storage device coupled to the one or more processors and having instructions stored thereon which, when executed by the one or more processors, cause the one or more processors to perform operations comprising: receiving, from a user interface displayed to the display device, an initial response data regarding a disease state. The initial response data is processed through an interface model to determine a series of next questions. The series of next questions is provided to the user interface. Subsequent response data for the series of next questions is received from the user interface. The initial response data and the subsequent response data are processed through a shared decision making engine to determine the treatment recommendations for the disease state from a plurality of treatment options for the disease state based on a weighted matrix, the weighted matrix including combinations of answers weighted according to relevance factors for treatment options for the disease state. The treatment recommendations for the disease state is provided to the user interface.

An aspect combinable with the general implementations, the treatment recommendations are determined based on a total weighted match score for each of the treatment options determined according to the weighted matrix, the initial response data and the subsequent response data.

In an aspect combinable with any of the previous aspects, the method or the operation comprise receiving clinical and research data regarding the disease state, and processing the clinical and research through an adjustment model to update the weighted matrix.

In an aspect combinable with any of the previous aspects, the interface model and the adjustment model each comprise deep neural networks.

In an aspect combinable with any of the previous aspects, the adjustment model is trained through machine learning with historical clinical and research data.

In an aspect combinable with any of the previous aspects, the interface model is trained through machine learning with data collected from user testing and simulated data.

In an aspect combinable with any of the previous aspects, the disease state is breast cancer or a predisposition to breast cancer.

In an aspect combinable with any of the previous aspects, the treatment options include lumpectomy, oncoplastic surgery, mastectomy, and breast reconstruction.

In an aspect combinable with any of the previous aspects, the user interface includes a chatbot.

Particular implementations of the subject matter described in this disclosure can be implemented so as to realize one or more of the following advantages. For example, the described shared decision making system can be employed to improve patient education, decrease a patient's anxiety, decrease decisional conflict, improve a patient's “buy-in” for a proposed treatment, appropriately set a patient's expectations, improve a patient's satisfaction with their treatment, and improve a patient's reported outcomes.

It is appreciated that methods in accordance with the present disclosure can include any combination of the aspects and features described herein. That is, methods in accordance with the present disclosure are not limited to the combinations of aspects and features specifically described herein, but also may include any combination of the aspects and features provided.

The details of one or more implementations of the present disclosure are set forth in the accompanying drawings and the description below. Other features and advantages of the present disclosure will be apparent from the description and drawings, and from the claims.

### BRIEF DESCRIPTION OF DRAWINGS

FIG. 1 depicts an example environment that can be employed to execute implementations of the present disclosure.

FIG. 2 schematically depicts an example system in accordance with implementations of the present disclosure.

FIGS. 3A-3I depict example user interfaces in accordance with implementations of the present disclosure.

FIG. 4 depicts a flow diagram of example processes that can be employed within a decision making system.

FIG. 5 depicts an example of a computing device and a mobile computing device that may be employed to execute implementations of the present disclosure.

### DETAILED DESCRIPTION

This disclosure generally describes a shared decision making system employed to provide treatment options for patients. The disclosure is presented to enable any person skilled in the art to make and use the disclosed subject matter in the context of one or more particular implementations. Various modifications to the disclosed implementations will be readily apparent to those skilled in the art, and the general principles defined in this application may be applied to other implementations and applications without departing from the scope of the disclosure. Thus, the present disclosure is not intended to be limited to the described or illustrated implementations, but is to be accorded the widest scope consistent with the principles and features disclosed in this application.

As an example context, thousands of new cases of breast cancer are diagnosed each year. And, surgery is an integral part of breast cancer treatment. Such surgery may include breast reconstruction after lumpectomy or mastectomy to prevent or minimize permanent deformity. Moreover, many patients are not offered the option of reconstruction. Of those patients that are made aware that they can have reconstruction, many are not informed of their reconstructive options. Additional, of the patients that are interested in breast reconstruction, for instance, only 43.3 percent make a “high-quality” breast reconstruction decision, which may be defined as having knowledge of at least 50 percent of the important facts and undergoing treatment concordant with one's personal preferences.

Adjuvant treatment for these patients may include chemotherapy, hormonal therapy, combined chemotherapy plus hormonal therapy, or observation alone. Making a treatment recommendation may involve framing questions, identifying management options and outcomes, collecting and summarizing evidence, and applying value judgments or preferences to arrive at an optimal course of action. Each step in this process can be conducted systematically (thus protecting against bias) or unsystematically (leaving the process open to bias). Treatment recommendations can be made based on the patient's risk of recurrence and the benefits and potential side effects of therapy.

In view of the foregoing, and as described in further detail herein, implementations of the present disclosure provide for a shared decision making system. The described system employs both machine learning and artificial intelligence in conjunction with a shared decision making engine to provide users (e.g., patients) a customized treatment recommendation for a specific clinical problem or disease state, such as surgical treatment options for breast cancer and breast reconstruction, that can be broken down into its component subtopics. Other disease states may include a predisposition to breast cancer (e.g., secondary to a gene mutation or strong family history) and where treatment options can include lumpectomy, oncoplastic surgery, mastectomy, and breast reconstruction. The described system may be incorporated in, for example, a digital health application relating to any disease state. The described shared decision making provides evidence-based approaches to addressing each clinical situation though a matrix weighted according to various peer reviewed literature for each of these and associated treatment options. An example context of breast cancer treatment is employed throughout this specification, however, the described system may be trained to provide treatment plans may be used for any disease state.

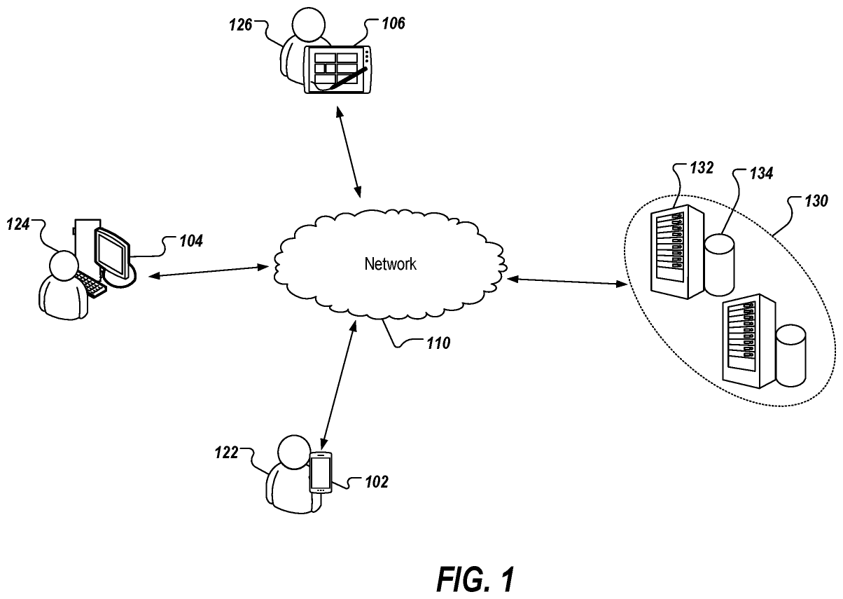

FIG. 1 depicts an example environment 100 that can be employed to execute implementations of the present disclosure. The example system 100 includes computing devices 102, 104, and 106, a back-end system 130, and a network 110. In some implementations, the network 110 includes a local area network (LAN), wide area network (WAN), the Internet, or a combination thereof, and connects web sites, devices (e.g., the computing devices 102, 104, 106) and back-end systems (e.g., the back-end system 130). In some implementations, the network 110 can be accessed over a wired and/or a wireless communications link. For example, mobile computing devices (e.g., the smartphone device 102 and the tablet device 106), can use a cellular network to access the network 110. In some examples, the users 122-126 interacting with a user interface that provides a customized treatment recommendation for a specific clinical problem.

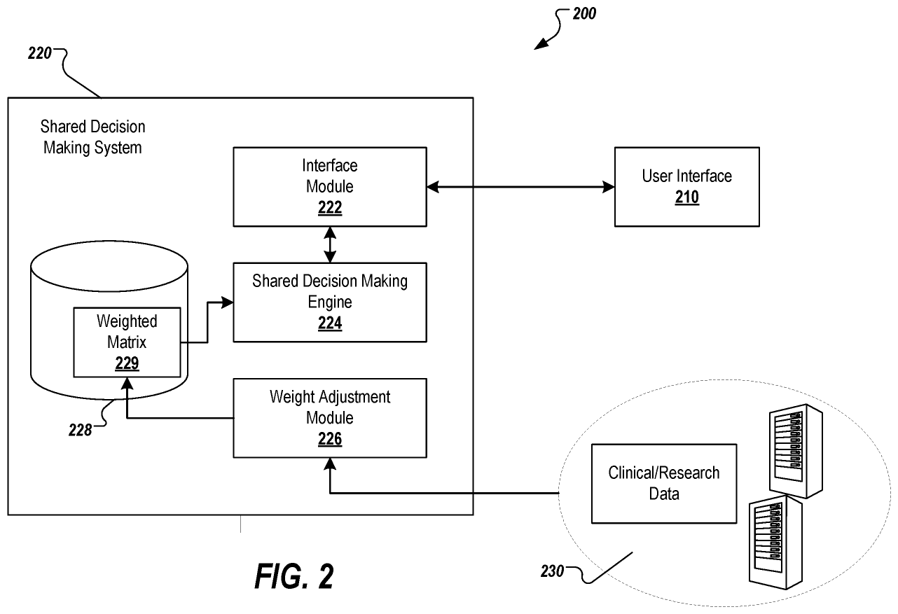

In the depicted example, the back-end system 130 includes at least one server system 132 and a data store 134. In some implementations, the at least one server system 132 hosts one or more computer-implemented services employed within the described DSO forecasting system, such as the modules described within architecture 200 (see FIG. 2), that users 122-126 can interact with using the respective computing devices 102-106. For example, the computing devices 102-106 may be used by respective users 122-126 to interact with a user interface that is provided through the back-end system 130. The user interface may provide the user a customized treatment recommendation for a specific clinical problem.

In some implementations, back-end system 130 may include server-class hardware type devices. In some implementations, back-end system 130 includes computer systems using clustered computers and components to act as a single pool of seamless resources when accessed through the network 110. For example, such implementations may be used in data center, cloud computing, storage area network (SAN), and network attached storage (NAS) applications. In some implementations, back-end system 130 is deployed using a virtual machine(s).

The computing devices 102, 104, 106 may each include any appropriate type of computing device such as a desktop computer, a laptop computer, a handheld computer, a tablet computer, a personal digital assistant (PDA), a cellular telephone, a network appliance, a camera, a smart phone, an enhanced general packet radio service (EGPRS) mobile phone, a media player, a navigation device, an email device, a game console, or an appropriate combination of any two or more of these devices or other data processing devices. In the depicted example, the computing device 102 is a smartphone, the computing device 104 is a desktop computing device, and the computing device 106 is a tablet-computing device. It is contemplated, however, that implementations of the present disclosure can be realized with any of the appropriate computing devices, such as those mentioned previously.

FIG. 2 schematically depicts an example system 200 in accordance with implementations of the present disclosure. The example system 200 may be implemented on a back-end system, such as back-end system 130 of FIG. 1. In the depicted example, the example system 200 includes a user interface 210, a shared decision making system 220, and clinical research data, provided though, for example, an application programming interface or a web crawl. In the depicted example, the shared decision making system 220 includes an interface module 222, a shared decision making engine 224, a weighted adjustment module 226, and a data store 228. In some examples, modules 222, 224, and 226 may be provided as one or more computer-executable programs executed by one or more computing devices (e.g., the back-end server system 130 of FIG. 1).

In some implementations, a user (e.g., users 122, 124, 126 of FIG. 1) interact with the shared decision making system 220 through the user interface 210. For example, the user interface 210 can be displayed by a computing device (e.g., the computing devices 102, 104, 106 of FIG. 1). The user interface 210 may be accessed through, for example, a browser application running on the computing device, or a mobile application. Mobile applications may include types of application software designed to run on a mobile device, such as a smartphone or tablet computer. The computing device may access the user interface 210 over a network (e.g., the network 110 of FIG. 1).

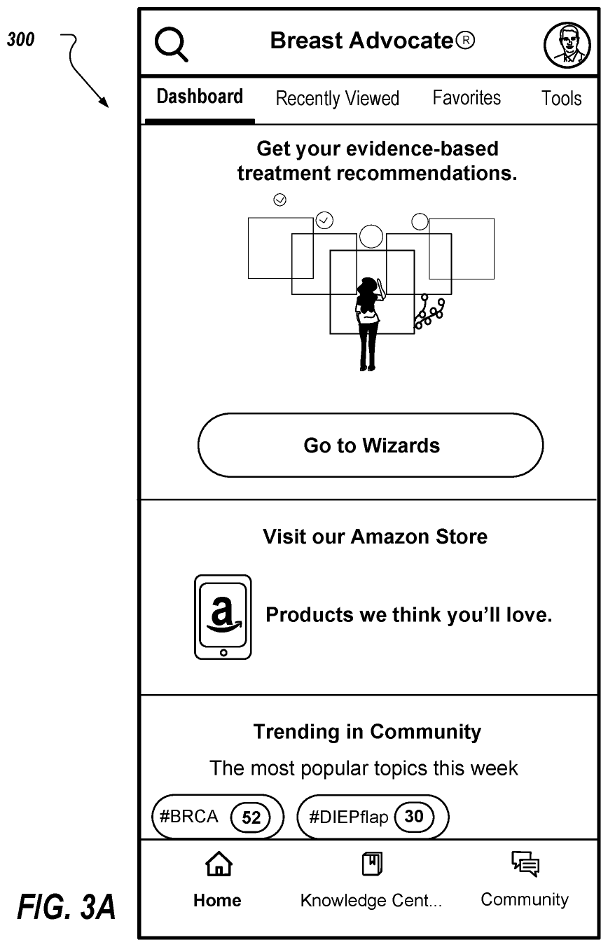

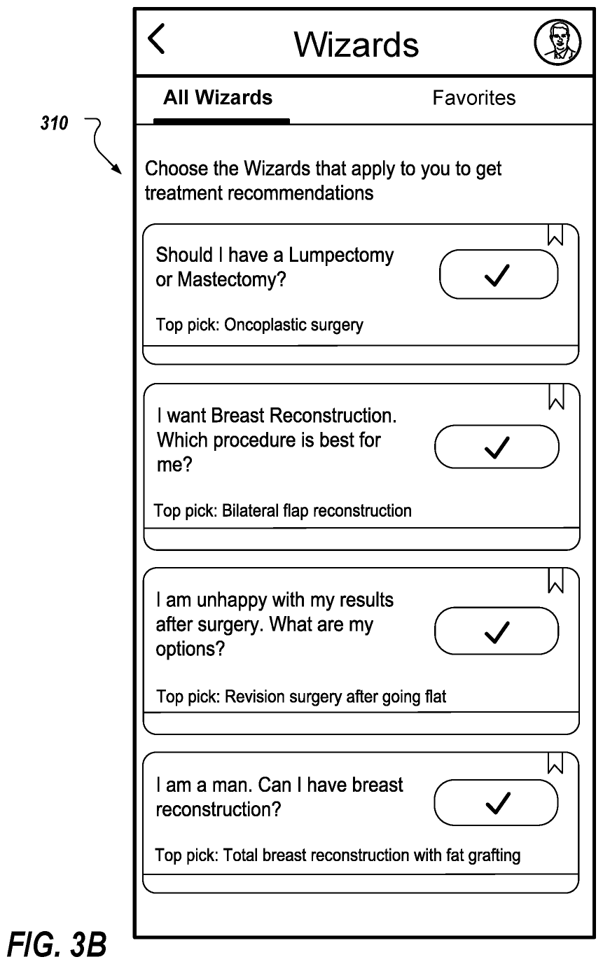

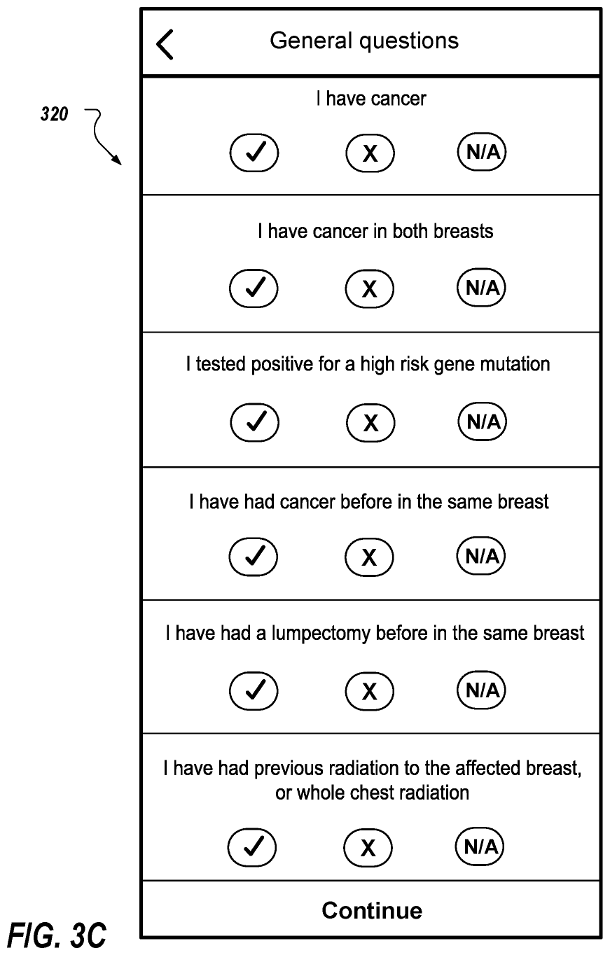

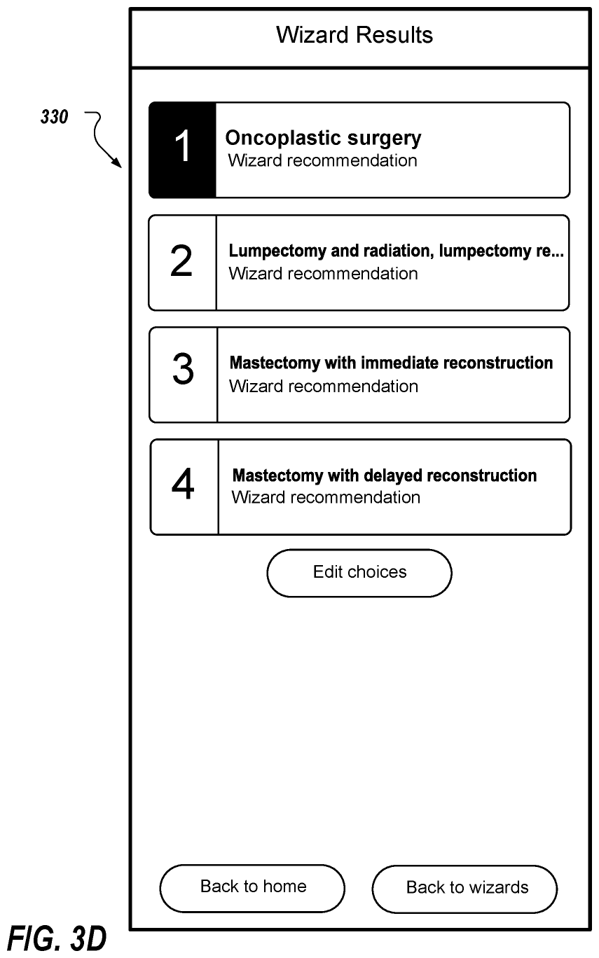

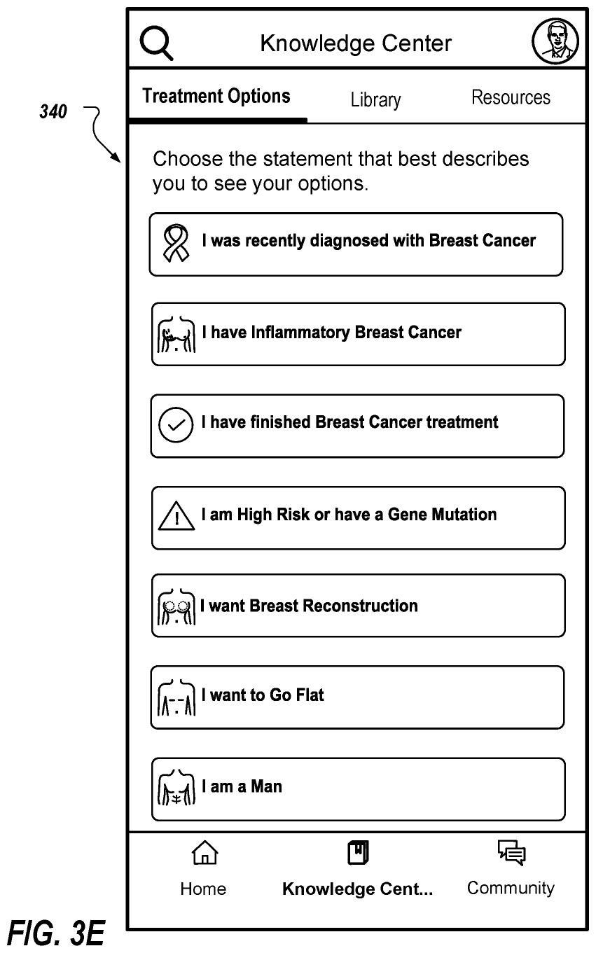

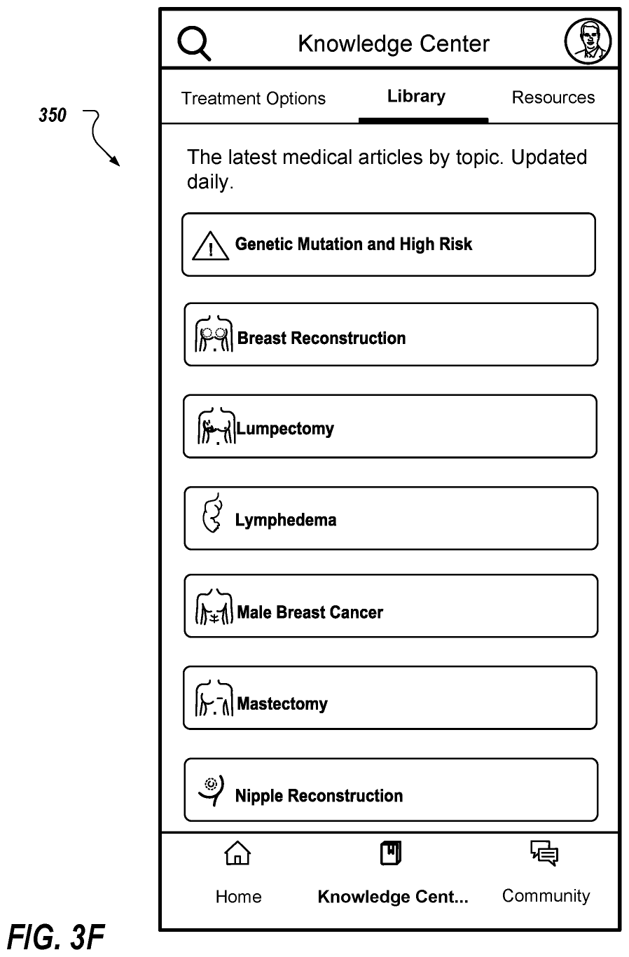

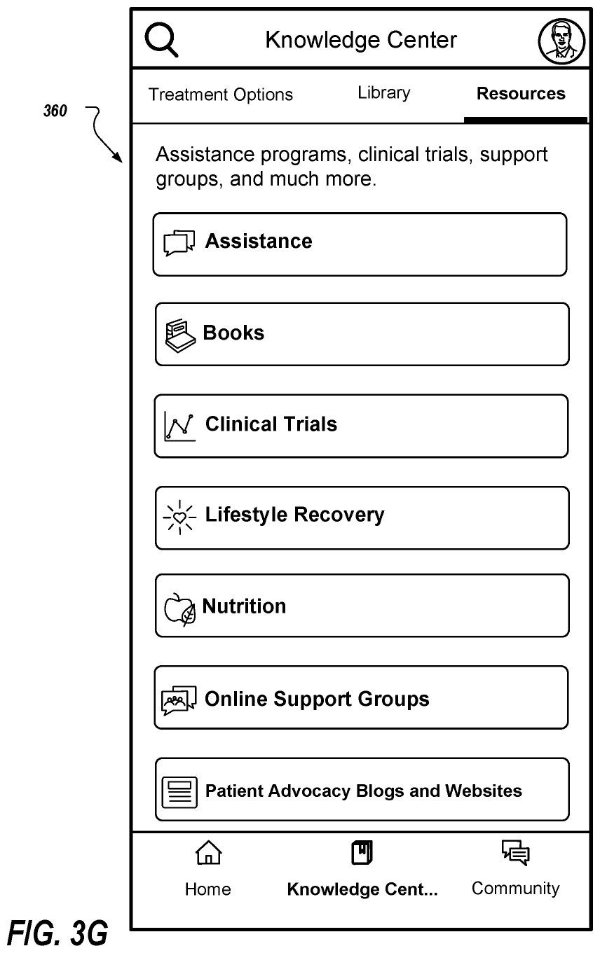

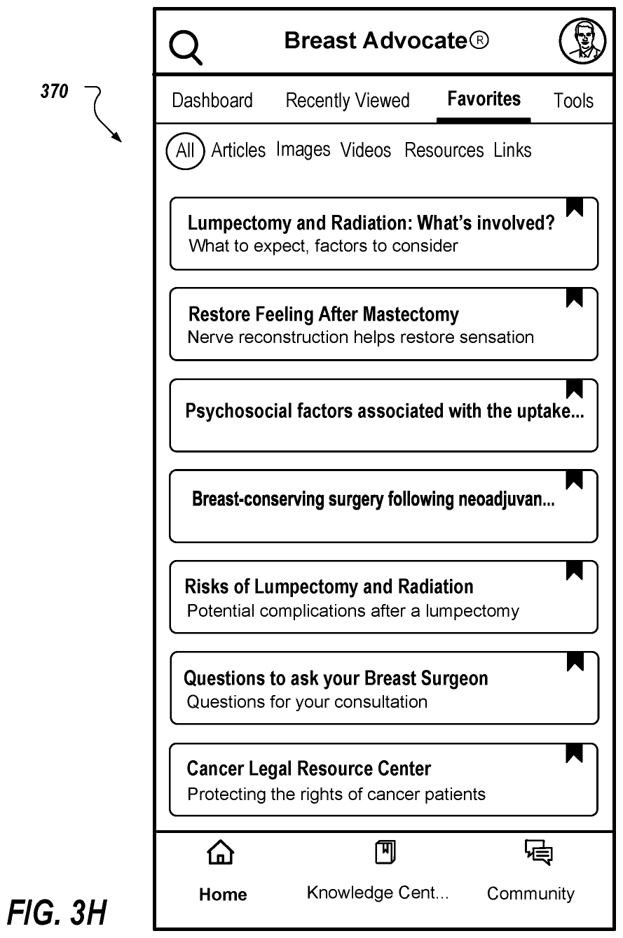

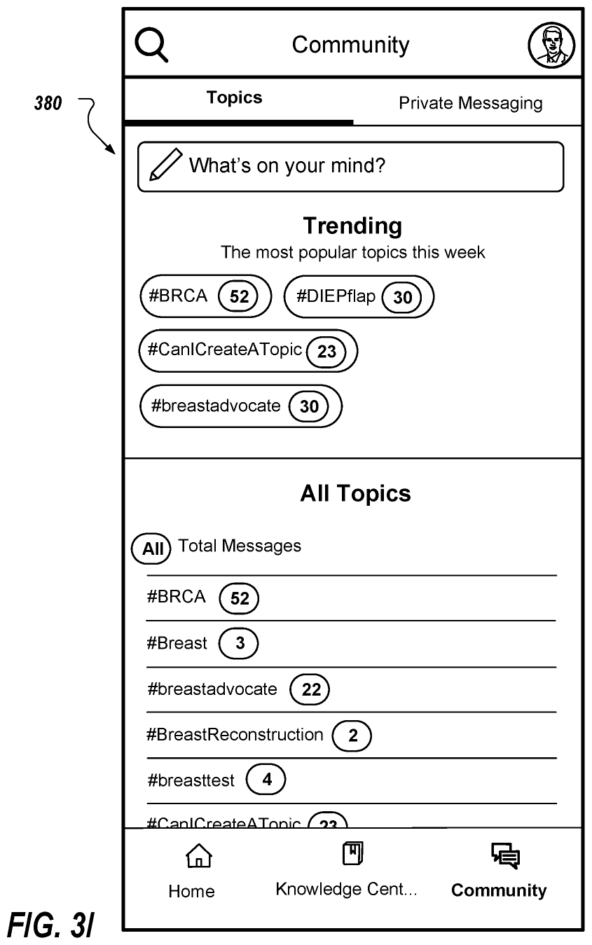

In some implementations, the user interface 210 may be provided as a graphical user interface (GUI). A GUI is generally presented as a field region in an image and may serve to facilitate interaction with a system, such as the decision making system 220. In some examples, a GUI may be provided through an application, such as a web browser or mobile application, executing on a computing device, and displayed to by a user. A GUI conveys information to the user and provides an interaction mechanism, through which the user might command the related system or computer, such as the intelligence reporting system 220. An example GUI for the user interface 210 are described in further detail herein with reference to FIGS. 3A-3I.

The user interface 210 enables users to interact with the shared decision making system 220. As described in further detail herein, the user interface 210 guides the user through a series of questions that prompts the user to select personal preferences. For example, through the user interface 210 the interface module 222 may receive anonymous user data, such as healthcare choices, demographics, and medical details. In some implementations, a user may be presented with a set of questions to assess a clinical picture (e.g., what is going on medically, the patient's diagnosis and history, current treatment plan, and so forth) and another set of questions to garner patient preferences and values.

The database 228 may be hosted by a back-end system (e.g., the back-end system 130 of FIG. 1.). The database 228 can be implemented using any appropriate database architecture, such as a relational database, an object-oriented database, one or more tables, and/or a distributed ledger, such as a blockchain. In some implementations, the database 228 is used to store the weighted matrix 229. The weighted matrix 229 may include combinations of answers to possible questions that can be presented to a user of the shared decision making system 200 through, for example, the user interface 210. In some implementations, the combinations of answers are weighted according to a relevance factor for each treatment option for a respective disease state or clinical problem. The weighted matrix 229 can be employed by the shared decision making engine 224 to determine outcomes, such as treatment recommendations, to users. In some implementations, each outcome determined by the shared decision making engine 224 is assigned a weighted match score determined according to the weighted matrix 229. After the questions are answered by a user, the shared decision making engine 224 may determine a total score for each treatment option. The scores are employed by the interface module 222 to provide users with top recommendation. In some implementations, the users may rank these treatment recommendations through the user interface 210.

In some implementations, the values in the weighted matrix 229 are updated through the weight adjustment module 226, which may search and index the clinical or research data 230. The clinical or research data 230 may include evidence-based literature. In some implementations, the weight values of the weighted matric 229 define how appropriate that a treatment would be in a clinical situation that includes the scenario represented by that question.

Implementations of the present disclosure can use machine learning techniques to train an algorithm(s) or model for use by the artificial intelligence (AI) interface module 222 and the AI weight adjustment module 226. For example, the interface module 222 may train an interface algorithm as to which questions to prompt the user for based on the users responses. Similarly, the weight adjustment module 226 may train an adjustment algorithm to update the weighted matrix 229 (see below) based on the clinical or research data.

The subject matter of machine learning includes the study of computer modeling of learning processes in their multiple manifestation. In general, learning processes include various aspects such as the acquisition of new declarative knowledge, the devilment of motor and cognitive skills through instruction or practice, the organization of new knowledge into general, effective representations, and the discovery of new facts and theories through observation and experimentations.

In some implementations, interface module 222 and the weight adjustment module 226 include or generates a machine learning model that has been trained to receive model inputs and to generate a predicted output for each received model input to execute one or more processes described in the present disclosure. In some implementations, the machine learning model is a deep model that employs multiple layers of models to generate an output for a received input. For example, the machine learning model may be a deep neural network. A deep neural network is a deep machine learning model that includes an output layer and one or more hidden layers that each apply a non-linear transformation to a received input to generate an output. In some cases, the neural network may be a recurrent neural network. A recurrent neural network is a neural network that receives an input sequence and generates an output sequence from the input sequence. In particular, a recurrent neural network uses some or all of the internal state of the network after processing a previous input in the input sequence to generate an output from the current input in the input sequence. In some other implementations, the machine learning model is a shallow machine learning model, e.g., a linear regression model or a generalized linear model.

In some implementations, interface module 222 and the weight adjustment module 226 can incorporate training data that is specific to a particular user or structure to generate the machine learning model(s). In some implementations, interface module 222 and the weight adjustment module 226 can obtain user specific training data during a training period (e.g., a training mode). A machine learning model may be trained with the training data. For example, the interface algorithm may be trained with user data collected from user testing and/or simulated data, while the adjustment algorithm may be trained with historical clinical or research data. In some implementations, interface module 222 and the weight adjustment module 226 can incorporate global training data (e.g., data sets) from a population of user or structures sources, such as sources accessible through the network 110 of FIG. 1. In some implementations, global training data can be related to users or research that is similar (e.g., demographically or otherwise) to the specific clinical problem for which the shared decision system making system 220 is programed to provided treatment recommendation, such a cancer diagnosis. In some aspects, the global training data can be crowd sourced.

When the user has completed the questions, in some examples, a “For you” section is displayed. This section may include customized recommended content and can be populated after a user uses the user interface 210 for the first time. In some implementations, the content shown in the “For you” section is specifically chosen based on the personal preferences the user expressed through their responses and determined through the shared decision making engine 224, which employs the weighted matrix 229. When a user updates their preferences (e.g., the question answer), the shared decision making engine 224 may determine new or additional content for the “For you” section based on the weighted matrix 229. In some implementations, the shared decision making engine 224 may determine new or updated content for the “For you” section when the weighted matric 229 is updated through the weighted adjustment module 226 (see below).

In some implementations, the interface module 222 employs the trained interface algorithm to select from a predefined set of initial questions based on the answers provided by the user. After responses are provided by the user for these initial questions, the interface module 222 may employ the trained interface algorithm to provide additional sections or groups of questions to the user. The response to these questions are provided to the shared decision making engine 224, which employs the weighted matrix 229 to determine treatment recommendations for each user. In some implementations, groups of questions may be shown or hidden from a user based on the user's responses to previous questions. The interface module 222, through the trained interface model, may determine whether to show or hide a question based on how relevant the questions is to the previously provided responses. For example, when a user wants breast reconstruction but does not want anything foreign in her body, the user may be provided with confirmatory questions to ensure consistency and then questions that focus on reconstructive techniques using, for example, the patient's own tissue, rather than implants.

In some implementations, the interface module may employ the trained interface algorithm within a chatbot. Chatbots (or “chatterbots”) are computer programs used to communicate information to users by mimicking conversations through audio or text. Chatbots may be employed in dialog systems, such as through user interface 210, to assist users by answering questions, providing a next group of questions, or providing help with navigation. Chatbots may also perform simple operations, such as accessing user information, as well as leveraging platform applications, such a website, database, or email service. Chatbot programming varies based on a variety of factors including the type of platform serviced, the operational logic used to build the chatbot, and the method(s) of communication supported. Common implementations of chatbots include rule-based logic, machine learning, and/or artificial intelligence. For example, some chatbots use sophisticated natural language processing (NLP) systems, but many simpler systems scan for keywords within the input, then pull a reply with the most matching keywords, or the most similar wording pattern, from a database.

The shared decision making system 220 employs the weight adjustment module 226 to crawl identified clinical data sources 230 for content (e.g., text), and retrieves relevant content. The clinical data sources 230 may include information regarding pathology (e.g., biopsy results), radiology (e.g., x-rays, ultrasounds, computed tomography (CT) scans, magnetic resonance imaging (MRI) scans, and so forth), laboratory result (e.g., blood test results, urine test results, and so forth), non-invasive tests (e.g., electrocardiogram, pulse oximetry, and so forth), measured vital signs, patient demographics, data input by patient, data uploads/synced (e.g., via electronic medical records (EMRs) or personal health devices, such as iWatch).

The weight adjustment module 226 may periodically make adjustments to the weighted matrix 229 based on the retrieved clinical data. In some implementations, one or more clinical data sources 230 to be searched/indexed by the weight adjustment module 226 can be predefined (e.g., by a system administrator). For example, a data clinical source can be identified based on a uniform resource locator (URL) assigned to the data source. A URL is a reference to a web resource that specifies a location of the data source on a computer network, as well as a mechanism for retrieving the data source.

It should be understood that, for illustrative purposes, FIG. 2 does not show other computer systems and elements which may be present when implementing the present disclosure. For example, the intelligence reporting system 220 may be deployed on a single computer system, or may be deployed in a computing environment that includes interconnected computer systems, on which data and programs are hosted or through an environment created by various virtual machines and services. Additional modules not illustrated in FIG. 2 may also be included and are to be considered within the scope of the present disclosure.

FIGS. 3A-3I depict example user interfaces in accordance with implementations of the present disclosure. The example user interfaces can be displayed as GUIs within the user interface 210 of FIG. 2. to enable a user to interact with the intelligence shared decision making system 220 of FIG. 2. In some implementations, the example GUIs are provided using one or more computer-executable programs executed by one or more computing devices (e.g., the backend system 103 of FIG. 1).

FIG. 3A depicts a dashboard screen 300 of an example GUI. As depicted, the dashboard screen 300 includes graphical form elements including header links to various pages within the GUI, such as recently viewed pages from the GUI, a favorites page, and a tools page. The dashboard screen 300 includes footer links to various pages within the GUI, such as the home page, a knowledge center page, and a community page. The dashboard screen 300 also includes a link to the treatment wizard. In some implementations, the dashboard screen 300 also includes dropdowns menus for a user's notes, their selected/assigned team, and recently viewed pages from the GUI. In some implementations, the drop down menus provide a list of relevant information and enables the user to select particular information.

FIGS. 3B-3D depict a wizard screens 310, 320, and 330 respectively of an example GUI respectively. The wizard screen 310 includes various links to topics and recommendations that a user may select to review information regarding and/or answer questions presented to the user. The wizard screen 320 includes example questions that may be presented to the user regarding a topic for which a user is attempting to obtain treatment recommendations. Such questions may be selected by the interface module 222 of FIG. 2. The wizard results screen 330 includes a list of recommended treatment options presented to the users based on the results from the shared decision making engine 224 of FIG. 2.

FIG. 3E depicts a treatment options categories screen 340 of an example GUI. The screen 340 includes links to various categories that a user can provide information to obtain a recommended treatment option. The screen 340 can also include a search query field to enable the user to enter a search query including one or more search terms to search for related content provide within the GUI.

FIGS. 3F-3G depicts a library screen 350 and resource screen 360 respectively. The library screen 350 and resource screen 360 each include links to various information (such as topical medication information and/or resource information) provided within the GUI. In some implementations, the library screen 350 and the resource screen 360 each include a search box to enable the user to enter a search query including one or more search terms to search for such content.

FIGS. 3H-3I depict additional interfaces and screens 370 and 380 that can be displayed in accordance with implementations of the present disclosure. Screen 370 is an example favorites screen. Screen 380 is an example community screen.

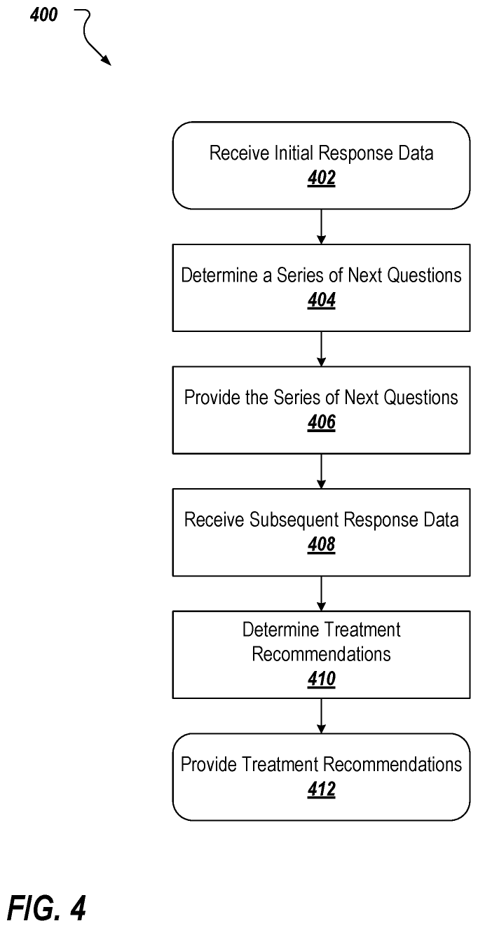

FIG. 4 depict a flow diagram of example processes 400 that can be employed within a decision making system, such as depicted in FIG. 2. For clarity of presentation, the description that follows generally describes process 400 in the context of FIGS. 1-3I, and 5. However, it will be understood that processes 400 may be performed, for example, by any other suitable system, environment, software, and hardware, or a combination of systems, environments, software, and hardware as appropriate. In some implementations, various steps of the processes 400 can be run in parallel, in combination, in loops, or in any order.

At 402, initial response data regarding the disease state is received from a user interface, initial response data regarding the disease state. In some implementations, the user interface include a chatbot. From 402, the process 400 proceeds to 404.

At 404, a series of next questions is determined by processing the initial response data through an interface model. In some implementations, the interface model is trained through machine learning with data collected from user testing and simulated data. From 404, the process 400 proceeds to 406.

At 406, the series of next questions are provided to the user interface. From 406, the process 400 proceeds to 408.

At 408, subsequent response data for the series of next questions is received from the user interface. From 408, the process 400 proceeds to 410.

At 410, treatment recommendations for the disease state is determined from a plurality of treatment options for the disease state based on a weighted matrix by processing the initial response data and the subsequent response data through a shared decision making engine. In some implementations, the weighted matrix includes combinations of answers weighted according to relevance factors for treatment options for the disease state. In some implementations, the treatment recommendations are determined based on a total weighted match score for each of the treatment options determined according to the weighted matrix, the initial response data and the subsequent response data. In some implementations, clinical and research data regarding the disease state is received and processing through an adjustment model to update the weighted matrix. In some implementations, the interface model and the adjustment model each comprise deep neural networks. In some implementations, the adjustment model is trained through machine learning with historical clinical and research data. From 410, the process 400 proceeds to 412.

At 412, the treatment recommendations for the disease state are provided to the user interface. In some implementations, the disease state is breast cancer, or a predisposition to breast cancer (e.g., secondary to a gene mutation or strong family history), and wherein the treatment options can include lumpectomy, oncoplastic surgery, mastectomy, and breast reconstruction. From 412, the process 400 ends.

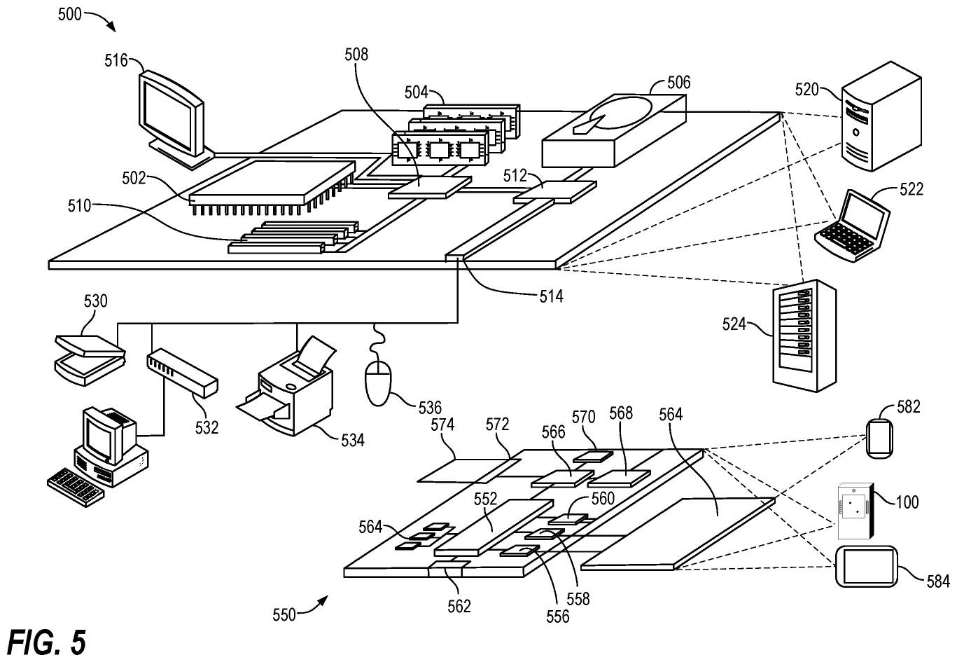

FIG. 5 depicts an example of a computing device 500 and a mobile computing device 550 that may be employed to execute implementations of the present disclosure. The computing device 500 is intended to represent various forms of digital computers, such as laptops, desktops, workstations, personal digital assistants, servers, blade servers, mainframes, and other appropriate computers. The mobile computing device 550 is intended to represent various forms of mobile devices, such as personal digital assistants, cellular telephones, smart-phones, AR devices, and other similar computing devices. The components shown here, their connections and relationships, and their functions, are meant to be examples only, and are not meant to be limiting.

The computing device 500 includes a processor 502, a memory 504, a storage device 506, a high-speed interface 508, and a low-speed interface 512. In some implementations, the high-speed interface 508 connects to the memory 504 and multiple high-speed expansion ports 510. In some implementations, the low-speed interface 512 connects to a low-speed expansion port 514 and the storage device 506. Each of the processor 502, the memory 504, the storage device 506, the high-speed interface 508, the high-speed expansion ports 510, and the low-speed interface 512, are interconnected using various buses, and may be mounted on a common motherboard or in other manners as appropriate. The processor 502 can process instructions for execution within the computing device 500, including instructions stored in the memory 504 and/or on the storage device 506 to display graphical information for a GUI on an external input/output device, such as a display 516 coupled to the high-speed interface 508. In other implementations, multiple processors and/or multiple buses may be used, as appropriate, along with multiple memories and types of memory. In addition, multiple computing devices may be connected, with each device providing portions of the necessary operations (e.g., as a server bank, a group of blade servers, or a multi-processor system).

The memory 504 stores information within the computing device 500. In some implementations, the memory 504 is a volatile memory unit or units. In some implementations, the memory 504 is a non-volatile memory unit or units. The memory 504 may also be another form of a computer-readable medium, such as a magnetic or optical disk.

The storage device 506 is capable of providing mass storage for the computing device 500. In some implementations, the storage device 506 may be or include a computer-readable medium, such as a floppy disk device, a hard disk device, an optical disk device, a tape device, a flash memory, or other similar solid-state memory device, or an array of devices, including devices in a storage area network or other configurations. Instructions can be stored in an information carrier. The instructions, when executed by one or more processing devices, such as processor 502, perform one or more methods, such as those described above. The instructions can also be stored by one or more storage devices, such as computer-readable or machine-readable mediums, such as the memory 504, the storage device 506, or memory on the processor 502.

The high-speed interface 508 manages bandwidth-intensive operations for the computing device 500, while the low-speed interface 512 manages lower bandwidth-intensive operations. Such allocation of functions is an example only. In some implementations, the high-speed interface 508 is coupled to the memory 504, the display 516 (e.g., through a graphics processor or accelerator), and to the high-speed expansion ports 510, which may accept various expansion cards. In the implementation, the low-speed interface 512 is coupled to the storage device 506 and the low-speed expansion port 514. The low-speed expansion port 514, which may include various communication ports (e.g., USB, Bluetooth, Ethernet, wireless Ethernet) may be coupled to one or more input/output devices. Such input/output devices may include a scanner 530, a printing device 534, or a keyboard or mouse 536. The input/output devices may also be coupled to the low-speed expansion port 514 through a network adapter. Such network input/output devices may include, for example, a switch or router 532.

The computing device 500 may be implemented in a number of different forms, as shown in the FIG. 5. For example, it may be implemented as a standard server 520, or multiple times in a group of such servers. In addition, it may be implemented in a personal computer such as a laptop computer 522. It may also be implemented as part of a rack server system 524. Alternatively, components from the computing device 500 may be combined with other components in a mobile device, such as a mobile computing device 550. Each of such devices may contain one or more of the computing device 500 and the mobile computing device 550, and an entire system may be made up of multiple computing devices communicating with each other.

The mobile computing device 550 includes a processor 552; a memory 564; an input/output device, such as a display 554; a communication interface 566; and a transceiver 568; among other components. The mobile computing device 550 may also be provided with a storage device, such as a micro-drive or other device, to provide additional storage. Each of the processor 552, the memory 564, the display 554, the communication interface 566, and the transceiver 568, are interconnected using various buses, and several of the components may be mounted on a common motherboard or in other manners as appropriate. In some implementations, the mobile computing device 550 may include a camera device(s) (not shown).

The processor 552 can execute instructions within the mobile computing device 550, including instructions stored in the memory 564. The processor 552 may be implemented as a chipset of chips that include separate and multiple analog and digital processors. For example, the processor 552 may be a Complex Instruction Set Computers (CISC) processor, a Reduced Instruction Set Computer (RISC) processor, or a Minimal Instruction Set Computer (MISC) processor. The processor 552 may provide, for example, for coordination of the other components of the mobile computing device 550, such as control of user interfaces (Uls), applications run by the mobile computing device 550, and/or wireless communication by the mobile computing device 550.

The processor 552 may communicate with a user through a control interface 558 and a display interface 556 coupled to the display 554. The display 554 may be, for example, a Thin-Film-Transistor Liquid Crystal Display (TFT) display, an Organic Light Emitting Diode (OLED) display, or other appropriate display technology. The display interface 556 may comprise appropriate circuitry for driving the display 554 to present graphical and other information to a user. The control interface 558 may receive commands from a user and convert them for submission to the processor 552. In addition, an external interface 562 may provide communication with the processor 552, so as to enable near area communication of the mobile computing device 550 with other devices. The external interface 562 may provide, for example, for wired communication in some implementations, or for wireless communication in other implementations, and multiple interfaces may also be used.

The memory 564 stores information within the mobile computing device 550. The memory 564 can be implemented as one or more of a computer-readable medium or media, a volatile memory unit or units, or a non-volatile memory unit or units. An expansion memory 574 may also be provided and connected to the mobile computing device 550 through an expansion interface 572, which may include, for example, a Single in Line Memory Module (SIMM) card interface. The expansion memory 574 may provide extra storage space for the mobile computing device 550, or may also store applications or other information for the mobile computing device 550. Specifically, the expansion memory 574 may include instructions to carry out or supplement the processes described above, and may include secure information also. Thus, for example, the expansion memory 574 may be provided as a security module for the mobile computing device 550, and may be programmed with instructions that permit secure use of the mobile computing device 550. In addition, secure applications may be provided via the SIMM cards, along with additional information, such as placing identifying information on the SIMM card in a non-hackable manner.

The memory may include, for example, flash memory and/or non-volatile random access memory (NVRAM), as discussed below. In some implementations, instructions are stored in an information carrier. The instructions, when executed by one or more processing devices, such as processor 552, perform one or more methods, such as those described above. The instructions can also be stored by one or more storage devices, such as one or more computer-readable or machine-readable mediums, such as the memory 564, the expansion memory 574, or memory on the processor 552. In some implementations, the instructions can be received in a propagated signal, such as, over the transceiver 568 or the external interface 562.

The mobile computing device 550 may communicate wirelessly through the communication interface 566, which may include digital signal processing circuitry where necessary. The communication interface 566 may provide for communications under various modes or protocols, such as Global System for Mobile communications (GSM) voice calls, Short Message Service (SMS), Enhanced Messaging Service (EMS), Multimedia Messaging Service (MMS) messaging, code division multiple access (CDMA), time division multiple access (TDMA), Personal Digital Cellular (PDC), Wideband Code Division Multiple Access (WCDMA), CDMA2000, General Packet Radio Service (GPRS). Such communication may occur, for example, through the transceiver 568 using a radio frequency. In addition, short-range communication, such as using a Bluetooth or Wi-Fi, may occur. In addition, a Global Positioning System (GPS) receiver module 570 may provide additional navigation- and location-related wireless data to the mobile computing device 550, which may be used as appropriate by applications running on the mobile computing device 550.

The mobile computing device 550 may also communicate audibly using an audio codec 560, which may receive spoken information from a user and convert it to usable digital information. The audio codec 560 may likewise generate audible sound for a user, such as through a speaker, e.g., in a handset of the mobile computing device 550. Such sound may include sound from voice telephone calls, may include recorded sound (e.g., voice messages, music files, etc.) and may also include sound generated by applications operating on the mobile computing device 550.

The mobile computing device 550 may be implemented in a number of different forms, as shown in FIG. 5. For example, it may be implemented the kiosk 100 described in FIG. 1. Other implementations may include a mobile device 582 and a tablet device 584. The mobile computing device 550 may also be implemented as a component of a smart-phone, personal digital assistant, AR device, or other similar mobile device.

Computing device 500 and/or 550 can also include USB flash drives. The USB flash drives may store operating systems and other applications. The USB flash drives can include input/output components, such as a wireless transmitter or USB connector that may be inserted into a USB port of another computing device.

Various implementations of the systems and techniques described here can be realized in digital electronic circuitry, integrated circuitry, specially designed application specific integrated circuits (ASICs), computer hardware, firmware, software, and/or combinations thereof. These various implementations can include implementation in one or more computer programs that are executable and/or interpretable on a programmable system including at least one programmable processor, which may be for a special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural, object-oriented, assembly, and/or machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a cathode ray tube (CRT) or liquid crystal display (LCD) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a GUI or a web browser through which a user can interact with an implementation of the systems and techniques described here), or any combination of such back end, middleware, or front end components. The components of the system can be interconnected by any form or medium of digital data communication, such as network 110 of FIG. 1. Examples of communication networks include a LAN, a WAN, and the Internet.

The computing system can include clients and servers. A client and server are generally remote from each other and typically interact through a communication network. The relationship of client and server arises by virtue of computer programs running on the respective computers and having a client-server relationship to each other.

Although a few implementations have been described in detail above, other modifications are possible. For example, while a client application is described as accessing the delegate(s), in other implementations the delegate(s) may be employed by other applications implemented by one or more processors, such as an application executing on one or more servers. In addition, the logic flows depicted in the figures do not require the particular order shown, or sequential order, to achieve desirable results. In addition, other actions may be provided, or actions may be eliminated, from the described flows, and other components may be added to, or removed from, the described systems. Accordingly, other implementations are within the scope of the following claims.

1. A computer-implemented method for providing treatment recommendations for a disease state, the method being executed by one or more processors and comprising:
receiving, from a user interface, initial response data regarding the disease state;
processing the initial response data through an interface model to determine a series of next questions;
providing the series of next questions to the user interface;
receiving, from the user interface, subsequent response data for the series of next questions;
processing the initial response data and the subsequent response data through a shared decision making engine to determine the treatment recommendations for the disease state from a plurality of treatment options for the disease state based on a weighted matrix, the weighted matrix including combinations of answers weighted according to relevance factors for treatment options for the disease state; and
providing the treatment recommendations for the disease state to the user interface.

2. The computer-implemented method of claim 1, wherein the treatment recommendations are determined based on a total weighted match score for each of the treatment options determined according to the weighted matrix, the initial response data and the subsequent response data.

3. The computer-implemented method of claim 1, comprising:
receiving clinical data and research data regarding the disease state; and
processing the clinical data and research data through an adjustment model to update the weighted matrix.

4. The computer-implemented method of claim 3, wherein the interface model and the adjustment model each comprise deep neural networks.

5. The computer-implemented method of claim 3, wherein the adjustment model is trained through machine learning with historical clinical and research data.

6. The computer-implemented method of claim 1, wherein the interface model is trained through machine learning with data collected from user testing and simulated data.

7. The computer-implemented method of claim 1, wherein the disease state is breast cancer or a predisposition to breast cancer.

8. The computer-implemented method of claim 1, wherein the treatment options include lumpectomy, oncoplastic surgery, mastectomy, and breast reconstruction.

9. The computer-implemented method of claim 1, wherein the user interface includes a chatbot.

10. One or more non-transitory computer-readable storage media coupled to one or more processors and having instructions stored thereon which, when executed by the one or more processors, cause the one or more processors to perform operations comprising:
receiving, from a user interface, initial response data regarding a disease state;
processing the initial response data through an interface model to determine a series of next questions;
providing the series of next questions to the user interface;
receiving, from the user interface, subsequent response data for the series of next questions;
processing the initial response data and the subsequent response data through a shared decision making engine to determine treatment recommendations for the disease state from a plurality of treatment options for the disease state based on a weighted matrix, the weighted matrix including combinations of answers weighted according to relevance factors for treatment options for the disease state; and
providing the treatment recommendations for the disease state to the user interface.

11. The one or more non-transitory computer-readable storage media of claim 10, wherein the treatment recommendations are determined based on a total weighted match score for each of the treatment options determined according to the weighted matrix, the initial response data and the subsequent response data.

12. The one or more non-transitory computer-readable storage media of claim 10, wherein the operations comprises:
receiving clinical data and research data regarding the disease state; and
processing the clinical data and research data through an adjustment model to update the weighted matrix.

13. The one or more non-transitory computer-readable storage media of claim 12, wherein the interface model and the adjustment model each comprise deep neural networks.

14. The one or more non-transitory computer-readable storage media of claim 12, wherein the adjustment model is trained through machine learning with historical clinical and research data.

15. A system, comprising:
a display device;
a one or more processors; and
a computer-readable storage device coupled to the one or more processors and having instructions stored thereon which, when executed by the one or more processors, cause the one or more processors to perform operations comprising:
receiving, from a user interface deployed to the display device, initial response data regarding a disease state;
processing the initial response data through an interface model to determine a series of next questions;
providing the series of next questions to the user interface;
receiving, from the user interface, subsequent response data for the series of next questions;
processing the initial response data and the subsequent response data through a shared decision making engine to determine treatment recommendations for the disease state from a plurality of treatment options for the disease state based on a weighted matrix, the weighted matrix including combinations of answers weighted according to relevance factors for treatment options for the disease state; and
providing the treatment recommendations for the disease state to the user interface.

16. The system of claim 15, wherein the interface model is trained through machine learning with data collected from user testing and simulated data.

17. The system of claim 15, wherein the disease state is breast cancer or a predisposition to breast cancer.

18. The system of claim 15, wherein the treatment options include lumpectomy, oncoplastic surgery, mastectomy, and breast reconstruction.

19. The system of claim 15, wherein the user interface includes a chatbot.

20. The system of claim 15, wherein the operations further comprise:
receiving clinical data and research data regarding the disease state; and
processing the clinical data and research data through an adjustment model to update the weighted matrix.
